(19) United States
(12) Patent Application Publication
(10) Pub. No.: US 2006/0159953 A1
(43) Pub. Date: Jul. 20, 2006

(54) Diarylamino substituted arylvinyl naphthalene compounds and a blue-light electroluminescent device having the compounds

(75) Inventors: Kuo-Wei Huang, Taipei (TW); Rai-Yi Chen, Pingchen City (TW); Chun-Liang Lai, Shuishang Hsiang (TW)

Correspondence Address: DELLETT & WALTERS, P. O. BOX 82788, PORTLAND, OR, 97282-0788, US

(73) Assignee: Labeltek Inc.

(21) Appl. No.: 11/040,274

(22) Filed: Jan. 20, 2005

(51) Int. Cl. C07C 211/58; H01L 51/54; H05B 33/14; C09K 11/06

(52) U.S. Cl. 428/690; 428/917; 313/504; 313/506; 257/E51.051; 564/429; 564/434

(57) ABSTRACT

Diarylamino arylvinyl naphthalene compounds, particularly used in blue-light organic light-emitting diodes with highly pure color, have the following representative formula: 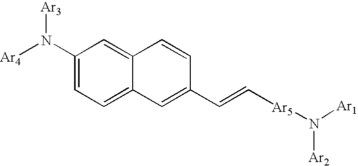
wherein Ar₁, Ar₂, Ar₃ and Ar₄ are identical or different substituents selected from the group consisting of phenyl, biphenyl, 1-naphthyl, 2-naphthyl, fluorophenyl, fluorobiphenyl, 1-fluoronaphthyl, 2-fluoronaphthyl, phenyl substituted by methyl or ethyl or isopropyl or tert-butyl or methoxy or ethoxy_, 1-naphthyl substituted by methyl or ethyl or isopropyl or tert-butyl or methoxy or ethoxy, 2-naphthyl substituted by methyl or ethyl, fluorophenyl substituted by methyl or isopropyl or tert-butyl or methoxy or ethoxy;
wherein “Ar₅” is one of the following substitutive groups consisting of: 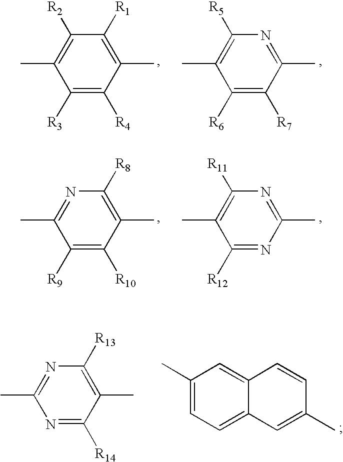
wherein the R₁ to R₁₄ are identical or different and are hydrogen,

### BACKGROUND OF THE INVENTION

1. Field of the Invention

The present invention relates to organic light emitting compounds, and more particularly to diarylamino substituted arylvinyl naphthalene compounds.

2. Description of Related Art

An organic light-emitting diode (OLED) is composed of a cathode, an anode and multiple electroluminescent layers. The electroluminescent layers contain special organic materials respectively being a hole-injecting material, a hole-transporting material, an electroluminescent material, an electron-conducting material and electron-injecting material. All electroluminescent layers are formed between the cathode and the anode by thermal vapor deposition or spin coating. When voltage is applied to the organic light-emitting diode, the anode generates electronic holes and the cathode generates electrons. When the electronic holes and the electrons combine with each other in the electroluminescent layers, light with a color is created and is emitted out of the organic light-emitting diode. The color of the light can be changed by using different organic electroluminescent materials.

Each electroluminescent layer is composed of a host material and a dopant material. In this host-dopant system, an exciton is generated when the hole and electron combine, and then energy is transferred to the dopant having excellent luminescent and stable capabilities to emit light from the electroluminescent layers. Thereby, the operational stability of the organic light-emitting diode is improved and non-luminescent energy decay is greatly reduced.

Because the luminescent efficiency and color purity are significantly related to the dopant material in the electroluminescent layers, selection of the dopant material is important. Several kinds of dopant materials in conventional organic light-emitting diodes have been developed.

1. Distyrylarylene (DSA) luminescent dopant containing a di-substituted amino group: The DSA dopant has luminescent efficiency 1˜2 lm/W but emits an impure blue-green color.

2. Stilbene (1,2-diphenyl ethylene) blue dopant with 4,4′-bis(2,2-diphenylvinyl)-1,1′-biphenyl: The stilbene dopant is blended with a host material, 4,4′-bis(2,2-diphenylvinyl)-1,1′-biphenyl (DPVBi) in the electroluminescent layers. The color of emitted light has color coordinates (0.146, 0.140). However, the stilbene dopant causes a low luminescent efficiency of about 2.1 lm/W and has overweight molecules that make the dopant easily decompose at high temperature during thermal vapor deposition.

3. Stilbene (1,2-diphenyl ethylene) blue dopant with a bianthracene host: the stilbene dopant blended with the bianthracene host has a luminescent efficiency up to 10.4 cd/A but has poor color purity.

4. Perylene blue dopant with an anthracene host: the perylene dopant blended with the anthracene host has a luminescent efficiency of 3.2 cd/A but has poor color purity with color coordinates of (0.154, 0.232).

The present invention has arisen to mitigate or obviate the disadvantages of the conventional dopant materials.

### SUMMARY OF THE INVENTION

The main objective of the present invention is to provide diarylamino substituted arylvinyl naphthalene compounds that serve as dopant materials to improve luminescent efficiency and color purity in the organic light-emitting diodes.

To achieve the foregoing objective, the diarylamino substituted arylvinyl naphthalene compounds have the following representative formula:

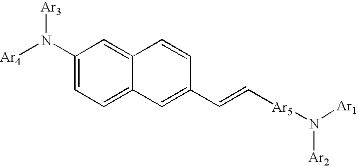

wherein Ar₁, Ar₂, Ar₃ and Ar₄ are identical or different substituents selected from the group consisting of phenyl, biphenyl, 1-naphthyl, 2-naphthyl, fluorophenyl, fluorobiphenyl, 1-fluoronaphthyl, 2-fluoronaphthyl, phenyl substituted by methyl or ethyl or isopropyl or tert-butyl or methoxy or ethoxy, 1-naphthyl substituted by methyl or ethyl or isopropyl or tert-butyl or methoxy or ethoxy, 2-naphthyl substituted by methyl or ethyl or isopropyl or tert-butyl or methoxy or ethoxy;

wherein “Ar₅” is one of the following substitutive groups comprising:

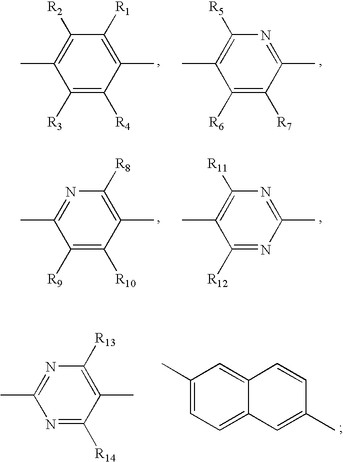

wherein the R₁ to R₁₄ are identical or different and are hydrogen, fluorine, straight-chain alkyl containing 1 to 4 carbon atoms, branched alkyl containing 3 to 4 carbon atoms, straight chain alkoxy containing 1 to 4 carbon atoms and branched alkoxy containing 3 to 4 carbon atoms.

By using the diarylamino substituted arylvinyl naphthalene compounds of the present invention as dopant materials for the electroluminescent medium in the at least one electroluminescent layer, the emitting efficiency of the organic light-emitting diode is increased and color purity is also improved.

Further benefits and advantages of the present invention will become apparent after a careful reading of the detailed description with appropriate reference to the accompanying drawings.

### DETAILED DESCRIPTION OF THE PREFERRED EMBODIMENT

Diarylamino substituted arylvinyl naphthalene compounds in accordance with the present invention are particularly used for highly efficient blue-light organic light-emitting diodes and have the following representative formula:

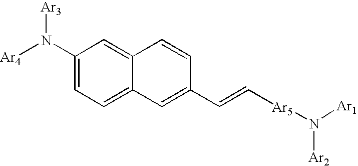

wherein Ar₁, Ar₂, Ar₃ and Ar₄ are identical or different substituents selected from the group consisting of phenyl, biphenyl, 1-naphthyl, 2-naphthyl, fluorophenyl, fluorobiphenyl, 1-fluoronaphthyl, 2-fluoronaphthyl, phenyl substituted by methyl or ethyl or isopropyl or tert-butyl or methoxy or ethoxy_{—}, 1-naphthyl substituted by methyl or ethyl or isopropyl or tert-butyl or methoxy or ethoxy, 2-naphthyl substituted by methyl or ethyl or isopropyl or tert-butyl or methoxy or ethoxy;

wherein “Ar₅” is chosen from the group consisting of the following: 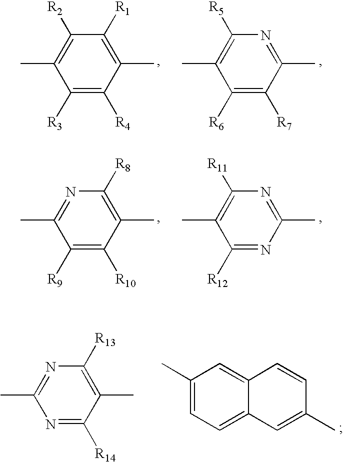
and

wherein the R₁ to R₁₄ are identical or different and are hydrogen, fluorine, straight-chain alkyl containing 1 to 4 carbon atoms, branched alkyl containing 3 to 4 carbon atoms, straight chain alkoxy containing 1 to 4 carbon atoms and branched alkoxy containing 3 to 4 carbon atoms.

The diarylamino substituted arylvinyl naphthalene compounds in the present invention is specifically of 2-[2-(diarylamino)aryl vinyl]-6-diarylamino naphthalene and have multiple practical embodiments in the following formulas:

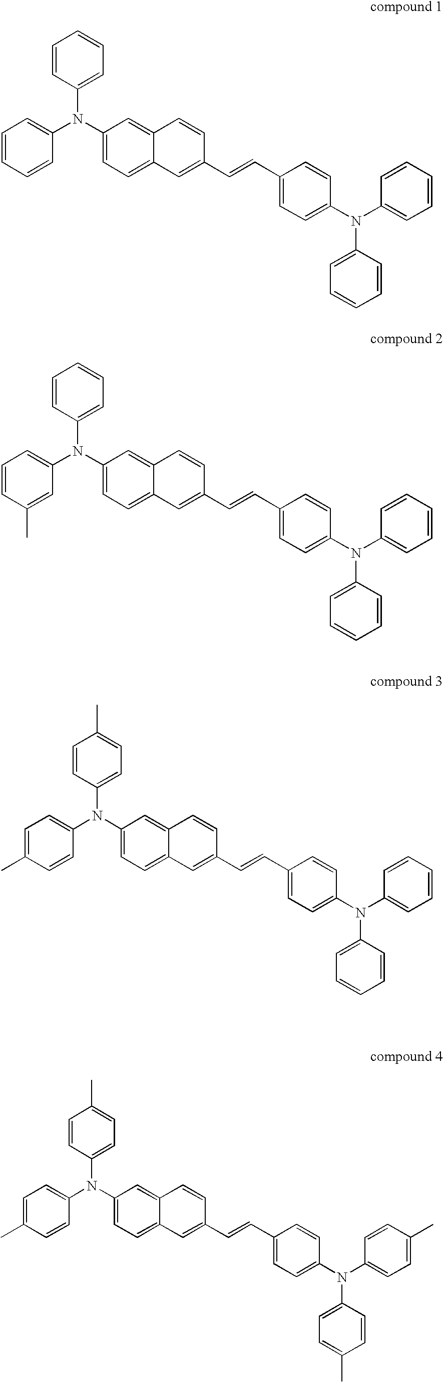

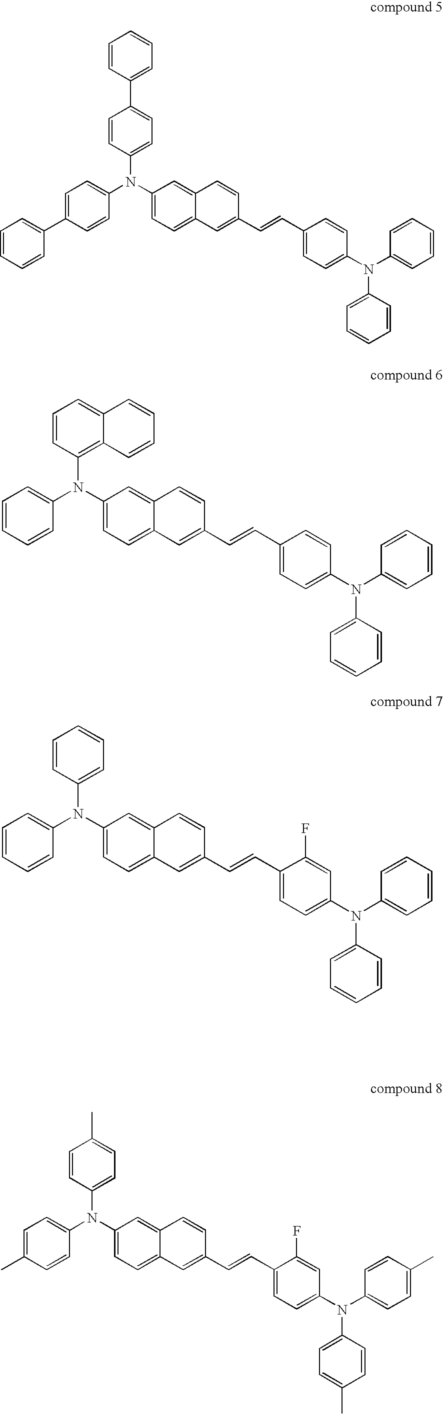

The diarylamino substituted arylvinyl naphthalene compounds corresponding to the representative formulas can be manufactured respectively by the following operational procedures of:

Reducing 6-bromo-2-methyl naphthoate by using lithium aluminium hydride in tetrahydrofuran solution;

substituting the alcohol group with bromine to prepare intermediate (6-bromo-2-bormomethyl) naphthalene by adding phosphorus tribromide;

substituing bromine with phosphonite by adding triethyl phosphite;

carrying out a Witting reaction with an aldehyde to form an intermediate product;

adding a diarylamine compound; and

coupling the diarylamine compound to the intermediate product to achieve the diarylamino substituted arylvinyl naphthalene compound by catalytic palladium.

The diarylamino substituted arylvinyl naphthalene compounds in the forgoing representative formulations are used in at least one electroluminescent layer in an organic light-emitting diode. When the organic light-emitting diode is manufactured, all organic materials including the host and the dopant are deposited on a transparent substrate in the form of layers by vacuum vapor deposition. The layers of the organic light-emitting diode are sequentially a substrate, an anode, a hole-injecting layer, a hole-transmitting layer, a luminescent layer, a electron-transmitting layer, a electron-injecting layer and a cathode or sequentially a substrate, a anode, a hole-transmitting layer, a luminescent layer, electron-transmitting layer, electron-injecting layer and a cathode.

Examples for Synthesizing the Aryl-substituted Diarylamino Arylvinyl Naphthalene Compounds:

### <EXAMPLE 1> COMPOUND I

First Act: Synthesization of Compound I

First, 200 g of 6-bromo-2-methyl naphthoate and 1500 ml of tetrahydrofuran were mixed in a reacting flask given a cool bath in a nitrogen atmosphere. In the cool bath, 21.5 g of lithium aluminium hydride was slowly added to the reacting flask to compose a mixture. The mixture was heated to 67° C., refluxed and stirred for two hours. Then, the mixture was bathed in cool water, and 100 g of water was added to the reacting flask to generate a white precipitation. The white precipitation was filtered out of the mixture and dried to obtain a white solid. The white solid was further mixed with water, stirred at room temperature, filtered and dried at 100° C. to obtain (6-bromo-2-naphthyl) methanol in a solid form. 169 g of the white solid was obtained (yield: 94.5%).

Second Act: Synthesization of Intermediate, (6-bromo-2-bromomethyl) Naphthalene.

169 g of (6-bromo-2-naphthyl) methanol and 1250 g of chloroform were placed in a reacting flask. phosphorus tribromide was dissolved in 180 ml of chloroform and slowly dropped into the reacting flask at room temperature in a nitrogen atmosphere to compose a mixture. The mixture was stirred for one-hour, and a white solid was precipitated after the mixture become transparent. Then, 750 ml of methanol was added to the mixture. After cold bathing and filtering, the solid obtained was further mixed with 750 ml of methanol solution again and stirred for 10 min. The solid was obtained by filtering the methanol solution and dried at 100° C. 110 g of (6-bromo-2bromomethyl) naphthalene was obtained (yield: 48.6%; purity: 99.2%).
Third Act: Synthesization of Compound A, 2-[4-(N,N-diphenylamino)styryl]-6-bromo-naphthalene

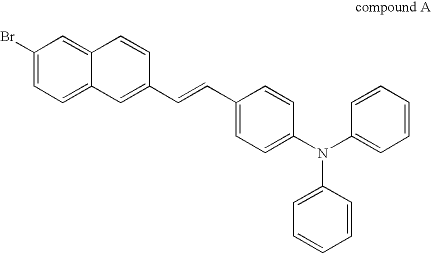

In ambient atmosphere, 50 g of (6-bromo-2-bromomethyl) naphthalene and 34.7 g of triethyl phosphite were placed into a reacting flask, heated to 130° C., stirred for 30 min, and then dried with vacuum pump. The reacting flask was cooled to room temperature to obtain a sticky liquid. The sticky liquid was mixed with 333 ml of dimethyl formamide and 77.2 g of potassium hydroxide, heated to 60° C., then mixed with 50.1 g of (N, N-diphenylamino)benzaldehyde, heated to 70° C. and stirred for 8 hours. 333 ml of methanol was further added to reacting flask to precipitate compound A in solid form that was obtained by filtering and drying at 110° C. for 1 hour. 74 g of compound A was obtained, and the compound A obtained had a melting point of 199° C. (DSC). (yield: 93%; purity: 95%)

Third Act: Synthesization of Compound 1

In a nitrogen atmosphere, 5 g of compound A, 2.14 g of diphenylamine, 1.2 g of sodium tert-butoxide, 19 mg of tris(dibenzylideneacetone)dipalladium, 8 mg of tri-tert-butylphosphine and 15 ml of xylene were mixed in a reacting flask to heat, reflux and stir for 1.5 hours. Then, 20 ml of methanol was added to the reacting flask to cool to 25° C. to precipitate compound 1. After filtering, the obtained compound 1 was further purified with toluene. 3.6 g of compound I was obtained (yield: 60%; purity: 95.8%)

Physical properties of the compound 1 obtained are: Tm 249° C. (DSC), Tg 82° C.(DSC), EIMS: m/e 564(M+) and 1H NMR (200 MHz, CDCl₃): δ 7.61˜7.73 (m, 4H), 7.37˜7.43 (m, 3H), 7.23˜7.30(m, 9H), 7.00˜7.15(m, 14H).

### <EXAMPLE 2> COMPOUND 2

The operational processes of synthesizing the compound 2 were the same as the processes in example 1 except the diphenylamine was substituted by 3-methyl diphenylamine. The compound 2 obtained had a purity of 92%.

Physical properties of the obtained compound II are: Tm 224° C. (DSC), Tg 77° C. and EIMS: m/e 578(M+).

### <EXAMPLE 3> COMPOUND 3

The operational processes of synthesizing the compound 3 were the same as the processes in example 1 except the diphenylamine was substituted by 4,4′-dimethyldiphenylamine. The obtained compound 3 had a purity of 94%.

Physical properties of the obtained compound III are: Tm 165° C. (DSC) and Tg 89° C.

### <EXAMPLE 4> COMPOUND 4

The operational processes of synthesizing the compound 4 were the same as the processes in example 1 except the diphenylamine was substituted by 4,4′-dimethyldiphenylamine and the compound A was substituted by a compound B as shown in the following.

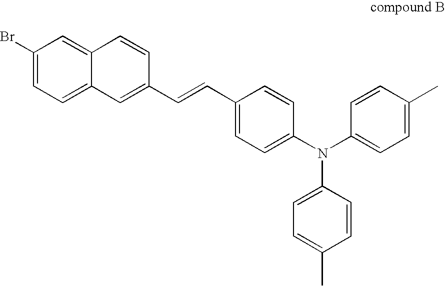

The obtained compound 4 had a purity of 98.8%. Physical properties of the obtained compound 4 are: Tm 240° C. (DSC) and Tg 96° C.

### <EXAMPLES 5 AND 6> COMPOUNDS 5 AND 6

The operational processes of synthesizing the compounds 5 and 6 were the same as the processes in example 1 except the diphenylamine was substituted by N-phenyl-1-naphthylamine and di(4-biphenyl)amine respectively for compound 5 and compound 6.

### <EXAMPLES 7 AND 8> COMPOUNDS 7 AND 8

A compound C as shown in the following formula was previously obtained.

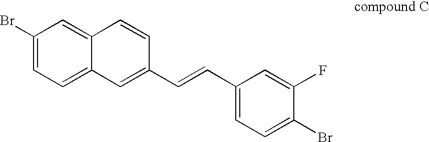

Synthesis of the compound C is the same as the synthesis of the compound A except the 4-(N,N-diphenylamino)benzaldehyde was substituted by 4-bromo-2-fluorobenzaldehyde.

The operational processes of synthesizing the compounds 7 and 8 were the same as the processes in example 1 except the quantity of the diphenylamine was doubled in example 7 and the diphenylamine was substituted by 4,4′-dimethyldiphenylamine in example 8.

Other derivatives in accordance with the diarylamino substituted arylvinyl naphthalene compounds in the present invention can be prepared by the operational processes in examples 1 or 7.

Examples of Organic Light-emitting Diodes Containing the Diarylamino Substituted Arylvinyl Naphthalene Compounds in the Present Invention:

### EXAMPLE 9

An ITO substrate with a resistivity of 20Ω/□ was mounted in a vapor-depositing machine. The vapor-depositing machine had a first quartz crucible containing N,N′-Di(4-biphenylyl)-N,N′-di(naphthalen-1-yl)-benzidine (NBB), a second quartz crucible containing 10,10′-di(4-biphenylyl)-9,9′-bianthryl (BH1), a third quartz crucible containing compound I, a fourth quartz crucible containing (tris-(8-hydroxyquinolinato)aluminum (Alq₃), a fifth crucible containing aluminum and a sixth crucible containing lithium fluoride.

Pressure in the vapor-depositing machine was reduced to 8×10⁻⁶ torr. The NBB in the first quartz crucible was heated to a vapor and deposited on the substrate as the hole-transporting layer to a thickness of 60 nm. Then, the BH1 in the second quartz crucible was heated to a vapor and deposited on the hole-transporting layer to a thickness of 30 nm to form the luminescent layer. Wherein, the light emitting layer further contained compound 1 that was 0.5% weight of the light emitting layer. An electron-transporting layer was made of Alq₃ using the same heating and deposition technique and formed on the luminescent layer. The sixth crucible was then heated to vaporize and deposit the lithium fluoride the lithium fluoride to a thickness of 0.8 nm on the electron-transporting layer to form an electron-injecting layer. Lastly, an aluminum cathode having a thickness of 150 nm was formed on the electron-injecting layer to achieve a first blue-light organic light-emitting diode.

When a direct current of 7.4 volts was applied to the first blue-light organic light-emitting diode, blue light was emitted with a light intensity of 2150 cd/m², a luminescent efficiency of 4.3 cd/A and a CIE coordinate of (X=0.145, Y=0.161).

### EXAMPLE 10

A second blue-light organic light-emitting diode has a structure and layer composition the same as the diode in example 9, except 3% weight of compound 2 was substituted for compound 1 in the light emitting layer.

When a 8.1 volt direct current was applied to the second organic light-emitting diode, blue light was emitted with a light intensity of 2390 cd/m², a luminescent efficiency of 4.78 cd/A and a CIE coordinate of (X=0.143, Y=0.169).

### EXAMPLE 11

A third blue-light organic light-emitting diode has the same structure and layer composition as the diode in example 9, except 1.5% weight of compound 2 was substituted for compound 1 in the light emitting layer.

When a 8.6 volt direct current was applied to the third organic light-emitting diode, blue light was emitted with a light intensity of 3077 cd/m², a luminescent efficiency of 6.16 cd/A and a CIE coordinate of (X=0.143, Y=0.167).

### EXAMPLE 12

A fourth blue-light organic light-emitting diode has the same structure and layer composition as the diode in example 9, except 3% weight of compound 3 was substituted for compound 1 in the light emitting layer.

When a 8.9 volt direct current was applied to the fourth organic light-emitting diode, blue light was emitted with a luminescent efficiency of 7.1 cd/A and a CIE coordinate of (X=0.147, Y=0.223).

### EXAMPLE 13

A fifth blue-light organic light-emitting diode has the same structure and layer composition as the diode in example 9, except 3% weight of compound 4 was substituted for compound 1 in the light emitting layer.

When a 9.2 volt direct current was applied to the second organic light-emitting diode, blue light was emitted with a luminescent efficiency of 8.2 cd/A and a CIE coordinate of (X=0.148, Y=0.229).

<Comparison Between the Examples of Organic Light-emitting Diodes>

A comparatively conventional organic light-emitting diode containing compound X as shown in the following formula in accordance with prior art was prepared.

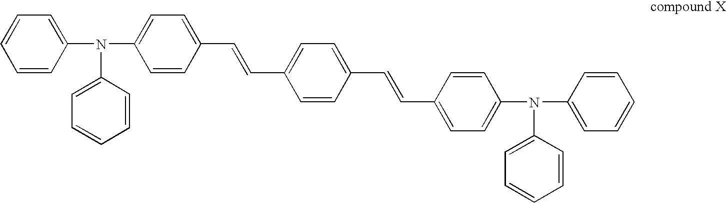

When a 9.2 volt direct current was applied to the conventional organic light-emitting diode, blue light was emitted with a light intensity of 10,000 cd/m², a luminescent efficiency of 11.2 cd/A and a CIE coordinate of (X=0.17, Y=0.30).

Properties of all organic light-emitting diodes are listed in the following table.

| | | | Luminescent | CIE |
|---|---|---|---|---|
| | | Dopant | efficiency | coordination |
| No | Compound | quantity | cd/A | (x,y) |
| Example 9 | I | 0.5% | 4.3 | (0.145, 0.161) |
| Example 10 | I | 3% | 4.78 | (0.143, 0.169) |
| Example 11 | II | 1.5% | 6.16 | (0.143, 0.167) |
| Example 12 | III | 3% | 7.1 | (0.147, 0.223) |
| Example 13 | IV | 3% | 8.2 | (0.148, 0.229) |
| Conventional | X | 3% | 11.2 | (0.17, 0.30) |

Structure of the organic light-emitting diodes: ITO/NBB/BH1+Dopant/Alq₃/LiF/Al

According to the foregoing table, the organic light-emitting diodes containing the diarylamino substituted arylvinyl naphthalene compounds in the present invention has higher color purity than the conventional diode.

The invention has been described in detail with particular reference to certain preferred embodiments. However, variations and modifications can be effected within the spirit and scope of the invention.

Although the invention has been explained in relation to its preferred embodiment, many other possible modifications and variations can be made without departing from the spirit and scope of the invention as hereinafter claimed.

What is claimed is:

1. Diarylamino substituted arylvinyl naphthalene compounds for a blue-light organic light-emitting diode, the diarylamino substituted arylvinyl naphthalene compounds having a representative formula of: 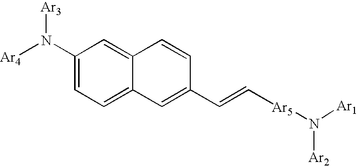
wherein Ar₁, Ar₂, Ar₃ and Ar₄ are identical or different substituents selected from the group consisting of phenyl, biphenyl, 1-naphthyl, 2-naphthyl, fluorophenyl, fluorobiphenyl, 1-fluoronaphthyl, 2-fluoronaphthyl, phenyl substituted by straight-chain alkyl containing 1 to 4 carbon atoms, phenyl substituted by branched alkyl containing 3 to 4 carbon atoms, phenyl substituted by straight chain alkoxy containing 1 to 4 carbon atoms, phenyl substituted by branched alkoxy containing 3 to 4 carbon atoms, 1-naphthyl substituted by straight-chain alkyl containing 1 to 4 carbon atoms, 1-naphthyl substituted by branched alkyl containing 3 to 4 carbon atoms, 1-naphthyl substituted by straight chain alkoxy containing 1 to 4 carbon atoms, 1-naphthyl substituted by branched alkoxy containing 3 to 4 carbon atoms, 2-naphthyl substituted by straight-chain alkyl containing 1 to 4 carbon atoms, 2-naphthyl substituted by branched alkyl containing 3 to 4 carbon atoms, 2-naphthyl substituted by straight chain alkoxy containing 1 to 4 carbon atoms, 2-naphthyl substituted by branched alkoxy containing 3 to 4 carbon atoms;
wherein “Ar₅” is chosen from the group consisting of the following groups: 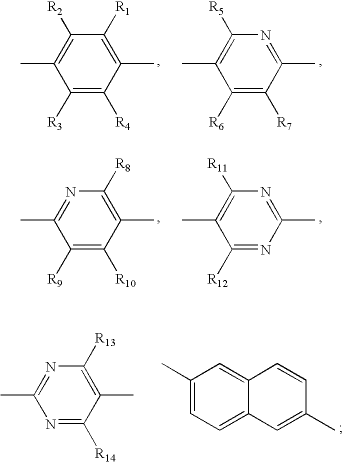
and
wherein the R₁ to R₁₄ are identical or different and are hydrogen, fluorine, straight-chain alkyl containing 1 to 4 carbon atoms, branched alkyl containing 3 to 4 carbon atoms, straight chain alkoxy containing 1 to 4 carbon atoms and branched alkoxy containing 3 to 4 carbon atoms.

2. The diarylamino substituted arylvinyl naphthalene compounds as claimed in claim 1, wherein the compounds formed from the general formula include a compound 1 that is:

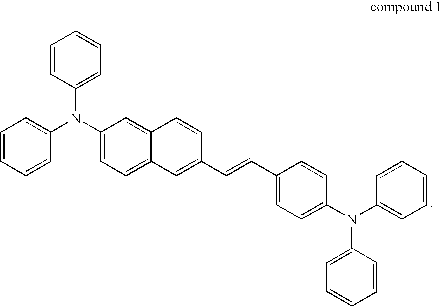

3. The diarylamino substituted arylvinyl naphthalene compounds as claimed in claim 1, wherein the compounds formed from the general formula include a compound 2 that is:

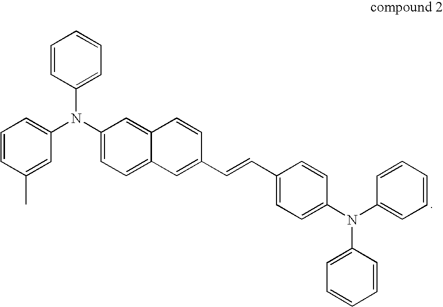

4. The diarylamino substituted arylvinyl naphthalene compounds as claimed in claim 1, wherein the compounds formed from the general formula include a compound 3 that is:

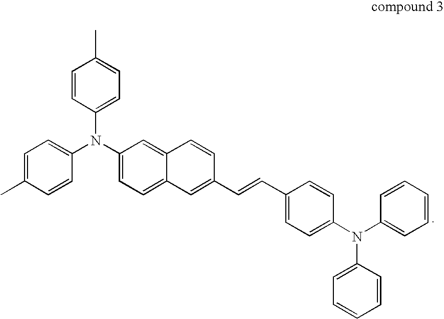

5. The diarylamino substituted arylvinyl naphthalene compounds as claimed in claim 1, wherein the compounds formed from the general formula include a compound 4 that is:

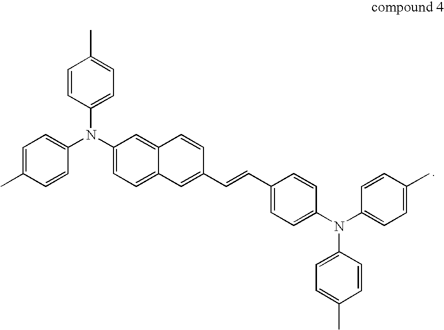

6. The diarylamino substituted arylvinyl naphthalene compounds as claimed in claim 1, wherein the compounds formed from the general formula include a compound 5 that is:

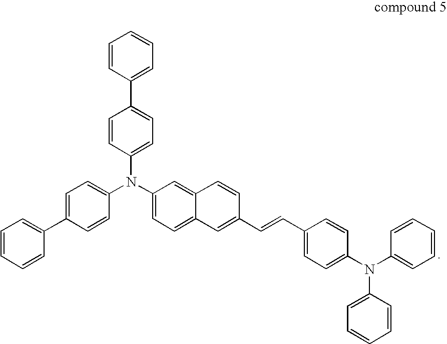

7. The diarylamino substituted arylvinyl naphthalene compounds as claimed in claim 1, wherein the compounds formed from the general formula include a compound 6 that is:

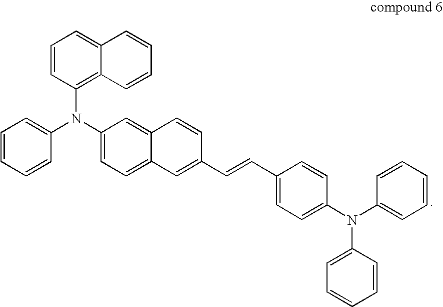

8. The diarylamino substituted arylvinyl naphthalene compounds as claimed in claim 1, wherein the compounds formed from the general formula include a compound 7 that is:

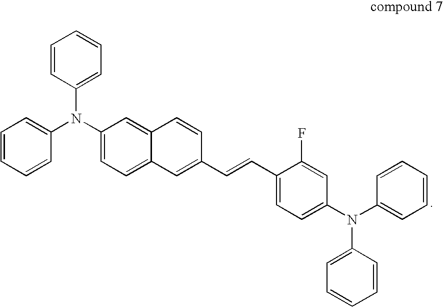

9. The diarylamino substituted arylvinyl naphthalene compounds as claimed in claim 1, wherein the compounds formed from the general formula include a compound 8 that is:

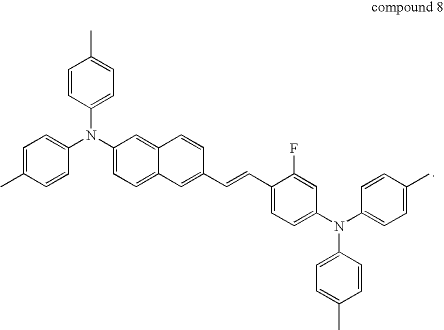

10. An organic light-emitting diode using at least one organic light emitting compound as claimed in claim 1, wherein the organic light-emitting diode comprises at least one luminescent layer having the at least one of the diarylamino substituted arylvinyl naphthalene compounds that have a representative formula of: 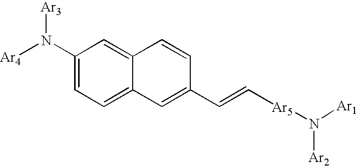
wherein Ar₁, Ar₂, Ar₃ and Ar₄ are identical or different substituents selected from the group consisting of phenyl, biphenyl, 1-naphthyl, 2-naphthyl, fluorophenyl, fluorobiphenyl, 1-fluoronaphthyl, 2-fluoronaphthyl, phenyl substituted by straight-chain alkyl containing 1 to 4 carbon atoms, phenyl substituted by branched alkyl containing 3 to 4 carbon atoms, phenyl substituted by straight chain alkoxy containing 1 to 4 carbon atoms, phenyl substituted by branched alkoxy containing 3 to 4 carbon atoms, 1-naphthyl substituted by straight-chain alkyl containing 1 to 4 carbon atoms, 1-naphthyl substituted by branched alkyl containing 3 to 4 carbon atoms, 1-naphthyl substituted by straight chain alkoxy containing 1 to 4 carbon atoms, 1-naphthyl substituted by branched alkoxy containing 3 to 4 carbon atoms, 2-naphthyl substituted by straight-chain alkyl containing 1 to 4 carbon atoms, 2-naphthyl substituted by branched alkyl containing 3 to 4 carbon atoms, 2-naphthyl substituted by straight chain alkoxy containing 1 to 4 carbon atoms, 2-naphthyl substituted by branched alkoxy containing 3 to 4 carbon atoms;
wherein “Ar₅” is chosen from the group consisting of the following: 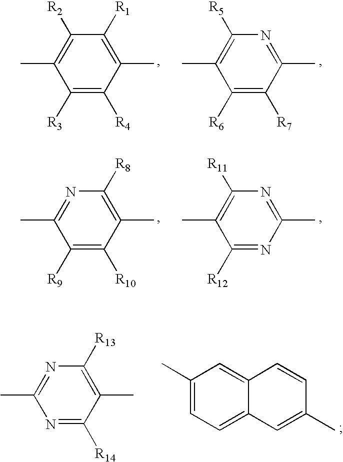
and
wherein the R₁ to R₁₄ are identical or different and are hydrogen, fluorine, straight-chain alkyl containing 1 to 4 carbon atoms, branched alkyl containing 3 to 4 carbon atoms, straight chain alkoxy containing 1 to 4 carbon atoms and branched alkoxy containing 3 to 4 carbon atoms.
